(19) United States
(12) Patent Application Publication
(10) Pub. No.: US 2026/0034315 A1
(43) Pub. Date: Feb. 5, 2026

(54) MICROFLUIDIC DEVICES AND METHODS OF PRODUCING

(71) Applicant: INVOX BELGIUM NV, Diepenbeek (BE)

(72) Inventor: Frank BARTELS, Hattingen (DE)

(21) Appl. No.: 19/099,094

(22) PCT Filed: Jul. 27, 2023
(86) PCT No.: PCT/EP2023/070867

§ 371 Date: Jan. 28, 2025

(30) Foreign Application Priority Data
Jul. 29, 2022 (EP) 22187883.8

(51) Int. Cl. A61M 11/04; B05B 1/26; B81B 1/00; B81C 1/00

(52) U.S. Cl. CPC A61M 11/041; B05B 1/26; B81B 1/004; B81C 1/00119; A61M 2207/00; B81B 2201/057; B81B 2203/0338; B81C 2201/0192

(57) ABSTRACT

The invention provides a microfluidic device (1), specifically a nozzle for an inhalation device, comprising at least two microfluidic structures (2A, 2A′), each of said structures (2A, 2A′) is located at a front end (1B) of the microfluidic device; characterized in that the microfluidic device (1) is made at least in part from a mono-crystalline material and wherein said front end (1B) of the microfluidic device (1) and the microfluidic structures (2A, 2A′) are aligned with the crystal orientation line and methods producing said devices and uses thereof.

### FIELD OF THE INVENTION

The invention is in the field of microfluidic devices, methods of producing microfluidic devices and uses of said devices. In particular, the invention relates to the production of microfluidic devices with high precision, in particular microfluidic devices for inhalation devices such as microfluidic nozzles.

### BACKGROUND OF THE INVENTION

Microfluidic devices are key parts of many medical and scientific devices. For example, Nebulizers and other aerosol generators utilize a plurality of microfluidic devices, in particular nozzles, valves and pump systems.

Nebulizers or other aerosol generators for liquids are known from the art since a long time ago. Amongst others, such devices are used in medical science and therapy.

There, they serve as inhalation devices for the application of active ingredients in the form of aerosols, i.e., small liquid droplets embedded in a gas. Such an inhalation device is known, e.g., from document EP 0 627 230 B1. Essential components of this inhalation device are a reservoir in which the liquid that is to be aerosolized is contained; a pumping unit for generation of a pressure being sufficiently high for nebulizing; as well as an atomizing device in the form of a nozzle.

In many inhalation devices, the nozzle is a microfluidic nozzle. In order to achieve a sufficiently homogenous and fine mist of liquid droplets, usually, relatively high pressures such as 10 bar, up to 1000 bar, are necessary. In order to keep the amount of vaporized liquid for each dose acceptably low, the nebulizing nozzle comprises usually one or several channels, each having a cross section only in the order of several μm², e.g., from 2 μm² to 200 μm². The channels are present in a nozzle body and are often fabricated using micro technological fabrication techniques such as micro etching, micro lithography and the like. However, these techniques are often targeted at hard and brittle materials such as silicon, glass or metal, and in order to avoid any undesired deformation of the nozzle when being subjected to said high pressures, the nozzle is often made from a very rigid material.

The parts require a high precision upon manufacturing. In particular, inhalers using impingement type nozzles require that the outflow channels of the microfluidic nozzle are precisely aligned, otherwise the resulting aerosol could be unsuitable for inhalation.

For fabrication of the nozzle from silicon or glass, often, a disc like substrate wafer is masked and irradiated with a large number of two-dimensional contours of the nozzles, or nozzle features such as the channels. Then, by way of selective etching, the contours, and in particular the channels, are vertically etched into the substrate, such that a wafer, carrying tens or hundreds of batch fabricated, semi-finished nozzles is present. In a separating step, the wafer is cut by way of sawing by a wafer saw into pieces which represent the individual nozzles.

The cutting process can be a source of error and result in a higher number of unusable nozzles or other microfluidic devices. FIGS. 1B illustrates a possible problem. FIG. 1A illustrates a wafer, comprising a plurality of impingement type nozzle bodies thereon, cut or sawed along an ideal separation line. FIG. 1B shows a wafer comprising a plurality of nozzles, which is cut at an angle different from the ideal separation line. This results in an offset in the channel exits, and thus the distance between the channel exits is different between each nozzle, resulting in different collision points for the aerosol jets, which could potentially affect the quality of the aerosol.

In WO 2019/180022 A1 a method for preparing a nozzle was described in which the nozzle exits were placed in a recess in the nozzle to compensate for errors of the cutting or sawing process.

However, using a recess is not suitable for every microfluidic device. As such, there is a need for an improved manufacturing method for microfluidic devices with high precision.

### SUMMARY OF THE INVENTION

In a first aspect, the invention relates to microfluidic devices, comprising at least two microfluidic structures, each of said structures is located at a front end of the microfluidic device; wherein the microfluidic device is made at least in part from a mono-crystalline material and wherein said front end of the microfluidic device and the microfluidic structures are aligned with the crystal orientation line or plane. In some embodiments, said microfluidic device is used in an inhalation device. In particular embodiments said microfluidic device is a nozzle for an inhalation device, preferably an impingement type nozzle.

In a second aspect, the present invention provides for a mono-crystalline wafer comprising a plurality of microfluidic devices according to the third aspect of the invention.

In a third aspect the invention relates to a method for producing a microfluidic device, comprising the steps of
a) providing a wafer substrate of a mono-crystalline material;
b) fabricating on said wafer substrate at least one microfluidic device comprising at least two microfluidic structures, wherein the microfluidic structures are aligned along the crystal orientation line or plane;
c) separating said microfluidic device (1) from the substrate by cleaving the wafer along the crystal orientation line or plane.

In further aspects the invention relates to the use of microfluidic devices according to the invention, in particular in inhalation devices.

### BRIEF DESCRIPTION OF THE DRAWINGS

FIGS. 1A and 1B show microfluidic devices on a wafer. FIG. 1A shows a cut along an ideal line, while FIG. 1B illustrates a non-ideal cut.

FIG. 2A shows options to cut wafer discs along different crystal orientations lines from mono-crystalline substrates.

FIG. 2B shows the cleavage behaviour of mono-crystalline wafers cut in and crystal orientation.

FIG. 2c depicts a unit cell of the diamond crystal structure.

FIG. 2D shows an overview of the diamond crystal structure comprising multiple unit cells, indicating crystal orientation lines therein.

FIG. 3 shows a simplified example for an impingement-type nozzle.

FIG. 4 shows a monocrystalline wafer with a plurality of microfluidic devices, specifically microfluidic nozzles etched thereon.

FIG. 5 shows a microfluidic device with a closing device, which comprises a pre-defined breaking point (top) and illustrates how said pre-defined breaking point can be used to precisely cleave a mono-crystalline wafer (bottom).

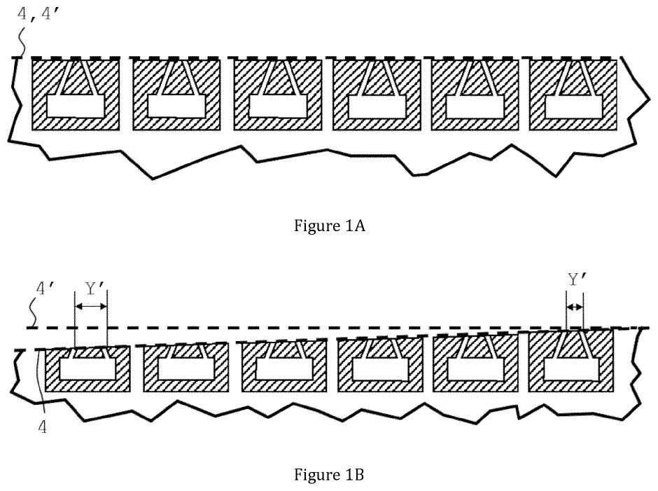

### DETAILED DESCRIPTION OF THE INVENTION

The inventors developed a new method to fabricate microfluidic devices with high precision utilizing mono-crystalline materials as a basis as well as improved microfluidic devices based on the method. Accordingly, in one aspect, the present invention relates to a microfluidic device, comprising at least two microfluidic structures, each of said structures is located at a front end of the microfluidic device; characterized in that the microfluidic device is made at least in part from a mono-crystalline material wherein said front end of the microfluidic device and the microfluidic structures are aligned with the crystal orientation line of the mono-crystalline material.

In a further aspect, the invention relates to method for producing microfluidic devices, wherein microfluidic structures are fabricated in a mono-crystalline wafer, and wherein the device is separated from said mono-crystalline wafer, for example, by breaking off or cutting, preferably by breaking off.

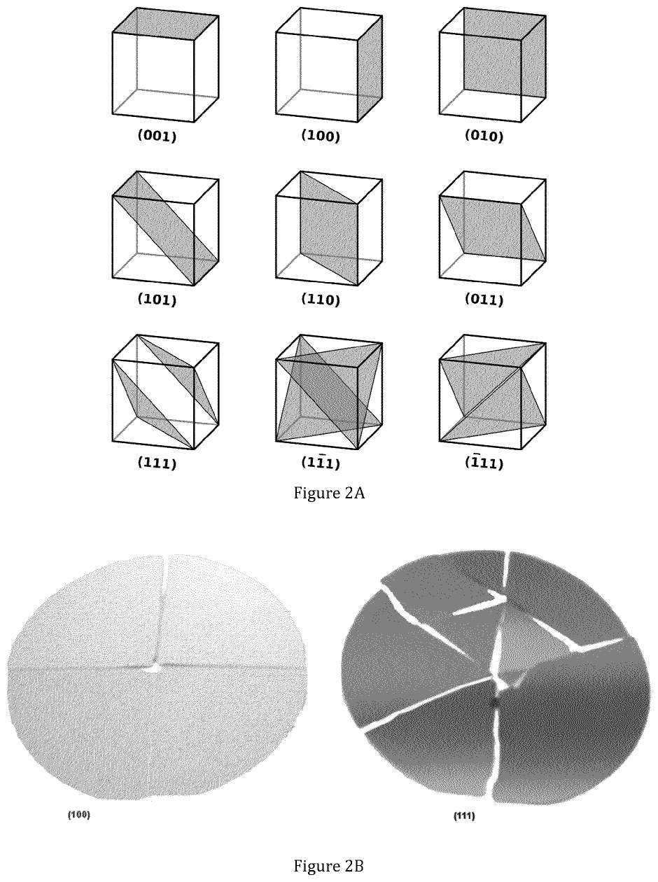

Mono-crystals can be obtained by the Czochralski process. Mono-crystals obtained by this process can be sliced into wafers using a wafer saw. By this high precision process, the wafer can be produced with a maximum 0.4° (degrees) derivation from the ideal orientation. The wafers can be sliced in different orientations with regard to the unit cell of the mono-crystal; see FIG. 2A. It has been found that the wafer discs show a different fracture behaviour depending on the orientation in which the mono-crystalline wafer was sliced from the mono-crystal; see FIG. 2B.

FIG. 2B shows that mono-crystalline wafers obtained from cuts in the [100] orientation show a distinct fracturing behaviour along the crystal orientation lines. The inventors found that microfluidic structures can be aligned with said crystal orientation lines to fabricate microfluidic devices with a high precision and reproducibility. This allows to reduce misalignments due to cutting or sawing processes and reduces rejects during production.

The term “crystal orientation line” as used in the context of the present invention refers to the borders of the unit cells of a crystal in the respective orientation of a mono-crystal. FIGS. 2C and 2D illustrate exemplary crystal-orientation lines of mono-crystals of the diamond structure. In top view of a wafer, the wafer corresponds to a single crystal plane, e.g., the [100] plane. As such, in top view, the [010] and [001] crystal orientation planes are essentially visible as single lines. Accordingly, in top view of the wafer, a crystal orientation line corresponds to a crystal orientation plane perpendicular to the plane in which the wafer is cut.

A mono-crystalline wafer produced in the orientation can therefore be cleaved along its crystal orientation lines in the or the orientation and will produce a clean and sharp cut along the orientation line.

Accordingly, the present invention also relates to a method for producing a microfluidic device comprising at least two microfluidic structures, the method comprising the steps of
a) providing a wafer substrate of a mono-crystalline material;
b) fabricating on said wafer substrate at least one microfluidic device comprising at least two microfluidic structures, each of said structures is located at a front end of the microfluidic device; wherein the microfluidic structures are aligned with the crystal orientation line;
c) separating said microfluidic device (1) from the substrate by cleaving the wafer along the crystal orientation line.

The monocrystalline wafer is cut in a specific crystal orientation so that the surface of the wafer corresponds to a crystal orientation plane.

The inventors found that aligning the microfluidic structures of the microfluidic devices with the crystal orientation and then cleaving along the crystal orientation line to separate the device from the substrate allows for a high precision fabrication with reduced rejects compared to cutting or sawing.

The term “microfluidic structure” as used in the context of the present invention refers to any kind of microfluidic structure. Preferred microfluidic structures include but are not limited to microfluidic chambers or reservoirs, and microfluidic channels. Microfluidic structures also include specific structures, such as microfluidic filter structures. Microfluidic structures also include microfluidic structures which may comprise additional components, which are not part of the mono-crystalline wafer substrate, such as microfluidic valves or mixing chambers.

In particular embodiments, the at least two microfluidic structures are microfluidic channels. In some embodiments, the at least two microfluidic structures are microfluidic channels comprising microfluidic channel exits. In more preferred embodiments, said microfluidic channel exits are located at the front end of the device.

In the context of the present invention the terms “aligned” or “aligning the microfluidic structures with the crystal orientation line” refer to positioning said microfluidic structures with reference to the crystal orientation line. The crystal orientation line, or in some embodiments, the crystal orientation lines are reference lines for the microfluidic device. This includes for example distances measured with reference to the microfluidic structure are measured with reference to the crystal orientation line. For example, the distance of two microfluidic channel exits is measured and based on the crystal orientation line. Alternatively, or additionally the angle of a channel may be measured relative to the crystal orientation line.

As a microfluidic wafer can be easily and exactly cleaved at a crystal orientation line, aligning the microfluidic structures or the front end of the microfluidic device with the crystal orientation line allows for high precision production with increased reproducibility and a reduced number of rejects.

Accordingly, in preferred embodiments of the invention, the front end of the microfluidic device is aligned or follows a crystal orientation line. Preferably, the front end of the device follows a crystal orientation line.

The mono-crystalline wafer is preferably a monocrystalline wafer obtained by cutting a mono-crystal from the Czochralski process. In particular embodiments, the mono-crystalline wafer is a mono-crystal obtained from a material that crystallizes in the diamond or perovskite crystal structure. Suitable materials are known to the skilled person and include but are not limited to mono-crystalline silicone, mono-crystalline germanium or mono-crystalline gallium arsenide. In preferred embodiments, the mono-crystalline material is mono-crystalline silicon.

For optimal cleavage properties the wafer is cut from a mono-crystal obtained by the Czochralski process and cut along the [100] crystal orientation. As shown in FIG. 2, wafers cut from mono-crystals in [100] orientation show an ideal cleavage behaviour along the crystal orientation lines.

Corresponding mono-crystalline wafers are commercially available, for example from Samsung, KR, or Micron, USA.

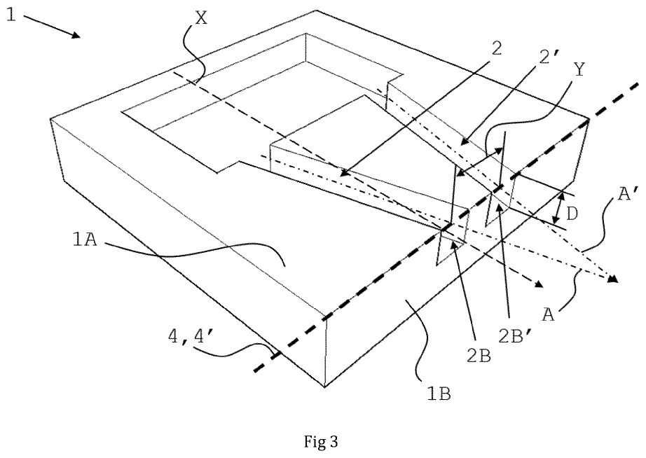

In many cases, suitable monocrystalline wafers are round, and do have one or two flat sides. As outlined in FIG. 4, said flat side (7) of the wafer (6) corresponds to a crystal orientation line of the wafer. All further corresponding crystal orientation lines will be perpendicular or parallel to said flat side of the wafer.

Accordingly, in a preferred embodiment of the invention the mono-crystalline wafer is a wafer in crystal orientation and the front end of the microfluidic device is aligned or corresponds to a [010] or [001] crystal orientation line. In preferred embodiments, the wafer is a round wafer comprising a flat side, which corresponds to a crystal orientation line. In some embodiments, the wafer comprises indentations indicating crystal orientation lines, which may serve as predetermined break points.

In some embodiments of the invention, the wafer substrate of step a) of the method of the invention is a wafer substrate cut from a monocrystal in [100] crystal orientation and said wafer substrate comprises a flat side corresponding to a crystal orientation line.

The wafer may be as thick or thin as necessary. In preferred embodiments, the thickness of the wafer is about 2 mm or less, for example between about 125 μm and about 2 mm. In some embodiments, the mono-crystalline wafer is less than 1.5 mm thick, for example it has a thickness of about 250 μm to about 1.5 mm. In some embodiments the thickness is 1 mm or less. In preferred embodiments, the thickness is about 750 μm or less, e.g., between about 300 μm and about 750 μm.

The microfluidic device may be fabricated on said wafer by any suitable means. The skilled person is aware of suitable means to fabricate a microfluidic device on a mono-crystalline wafer. Said methods may include chemical or mechanical methods. In some embodiments, the microfluidic device is fabricated by etching the microfluidic device into the mono-crystalline wafer. In some embodiments, the microfluidic device is fabricated by laser etching or laser ablation. In different embodiments, the microfluidic device is fabricated by sand blasting. In some embodiments, the microfluidic device is fabricated by micro lithography. In some embodiments, the microfluidic device is fabricated by a combination of different methods, e.g., a combination of etching and laser ablation.

Aside from the front end, which is aligned with or corresponds to a crystal orientation line, the microfluidic device may comprise at least one back end and at least one side end. The microfluidic device may in general have any shape, provided at least one side, i.e., said front end, is aligned with a crystal orientation line.

In preferred embodiments, the microfluidic device is rectangular, and comprises a front end, a back end, and two side ends. In more preferred embodiments, said back end is aligned to a crystal orientation line parallel to the front end of the microfluidic device and each side end is aligned or corresponds to a further crystal orientation line perpendicular to the back-end crystal orientation line. In such an embodiment, the method preferably comprises the step of:

Fabricating on said monocrystalline wafer at least one microfluidic device comprising at least two microfluidic structures, wherein the microfluidic structures are aligned along the crystal orientation; and wherein the front end and back end of the device are parallel and are aligned and correspond each to a crystal orientation line; optionally wherein the side ends are perpendicular to the front and back end, and are aligned and correspond each to crystal orientation lines perpendicular to the crystal orientation lines of the front and back end.

The method is also suitable for mass production of microfluidic devices with high precision. As such, the method may comprise fabricating a plurality of microfluidic devices on a single mono-crystalline wafer. The microfluidic devices may or may not be identical. Preferably, the monocrystalline wafer comprises a plurality of identical microfluidic devices, which are aligned in parallel, and their respective back ends and side ends are aligned with crystal orientation lines to allow for easy separation; see for example FIG. 4.

Accordingly, in some embodiments the method of the invention is a method for producing a plurality of microfluidic devices, each device comprising at least two microfluidic structures, the method comprising the steps of
a) providing a wafer substrate of a mono-crystalline material;
b) fabricating on said wafer substrate a plurality of microfluidic devices, each comprising at least two microfluidic structures, each of said structures is located at a front end of the corresponding microfluidic devices; wherein the microfluidic structures of each device are aligned with a crystal orientation line;
c) separating said microfluidic devices (1) from the substrate by cleaving the wafer along the crystal orientation lines.

In preferred embodiments, the wafer substrate of step a) of the method of the invention is a wafer substrate cut from a mono-crystalline material in crystal orientation and said wafer substrate comprises a flat side corresponding to a crystal orientation line.

In preferred embodiments of the invention, the front end of each of the plurality of microfluidic devices is aligned with a crystal orientation line.

Accordingly, in some embodiments of the invention, the wafer substrate is a wafer substrate as defined above and step b) of the method comprises: fabricating on said wafer substrate a plurality of microfluidic devices, each microfluidic device comprising at least two microfluidic structures, each of said structures is located at a front end of the respective microfluidic device; wherein the microfluidic structures are aligned with the crystal orientation line; and wherein the front end of at least one of said at plurality of microfluidic devices is aligned or preferably corresponds to the flat side of the mono-crystalline wafer substrate.

The method of the invention is particularly suitable for the mass production of rectangular microfluidic devices. Accordingly, in some embodiments, the invention relates to a method as defined above for producing a plurality of microfluidic devices, wherein said microfluidic devices comprise at least two microfluidic structures and each microfluidic device comprises a front end, a back end and two side ends. In preferred embodiments said back end is parallel to the front end of the device and said side ends are perpendicular to said front end and back end.

Therefore, in some embodiments, the invention relates to a method for producing a plurality of microfluidic devices as defined above, wherein each microfluidic device comprises a front end, a back end and two side ends, wherein the back end of a microfluidic device is parallel to the front end of the device and the side ends of a microfluidic device are perpendicular to the front end and back end of the microfluidic device. the method comprising the steps of:
a) providing a wafer substrate of a mono-crystalline material;
b) fabricating on said wafer substrate a plurality of microfluidic devices, each comprising at least two microfluidic structures, each of said structures is located at a front end of the respective microfluidic device; wherein the microfluidic structures are aligned with a crystal orientation line; wherein the plurality of microfluidic devices is fabricated in a checkerboard patten, wherein the microfluidic devices are positioned parallel to each other, in such a way that a first line of microfluidic devices comprises front ends aligned with a crystal orientation line, parallel back ends aligned with a different parallel crystal orientation line, and side ends aligned with perpendicular crystal orientation lines, wherein the side end of one microfluidic device is next to the side end of a further microfluidic device, and the back end of a microfluidic device is next to a front end of a microfluidic device;
c) separating the microfluidic devices (1) from the substrate and each other by cleaving the wafer along the crystal orientation lines.

The microfluidic devices may be identical or different microfluidic devices. Preferably, the microfluidic devices are identical.

The microfluidic device or devices can be cleaved by any suitable method from the wafer. The skilled person is aware of suitable methods to cleave the wafer at a crystal orientation line. Suitable method include cutting and breaking off or a combination thereof. In preferred embodiments, cleaving comprises breaking the microfluidic device off the microfluidic wafer at the crystal orientation line. Said breaking off may be manually or with a machine. In order to improve precision of the cleaving of the mono-crystalline wafer, said wafer may be covered with a plate comprising predetermined breaking points or breaking lines adapted to the wafer, wherein the breaking points or breaking lines are adapted to the crystal orientation lines of the mono-crystalline wafer. In such an embodiment, the plate with predetermined breaking points or breaking lines would be cleaved, in particular broken, together with the mono-crystalline wafer.

In some embodiments, the wafer may include predetermined breaking lines or breaking points. The wafer may be slightly chipped or indented at predetermined portions to create predetermined breaking points. It is also possible to add predetermined breaking points during the process of fabricating the microfluidic device into the wafer. For example, predetermined breaking points or breaking lines may be etched into the wafer. Such predetermined breaking points or breaking lines may be fabricated by the same method as the microfluidic device, e.g., by etching, in particular laser etching, laser ablation, sand blasting or micro lithography.

In some embodiments, the microfluidic devices are covered with a closing device before or after cleaving. Said closing device preferably seals and closes the microfluidic structures. In some embodiments, said closing device corresponds to a lid. In some embodiments said closing device corresponds to a cover.

The closing device may be permanently attached to the microfluidic device, for example by using glue or atomic bonding. In some embodiments, the closing device is placed on the microfluidic device and held in place by separate holding structures.

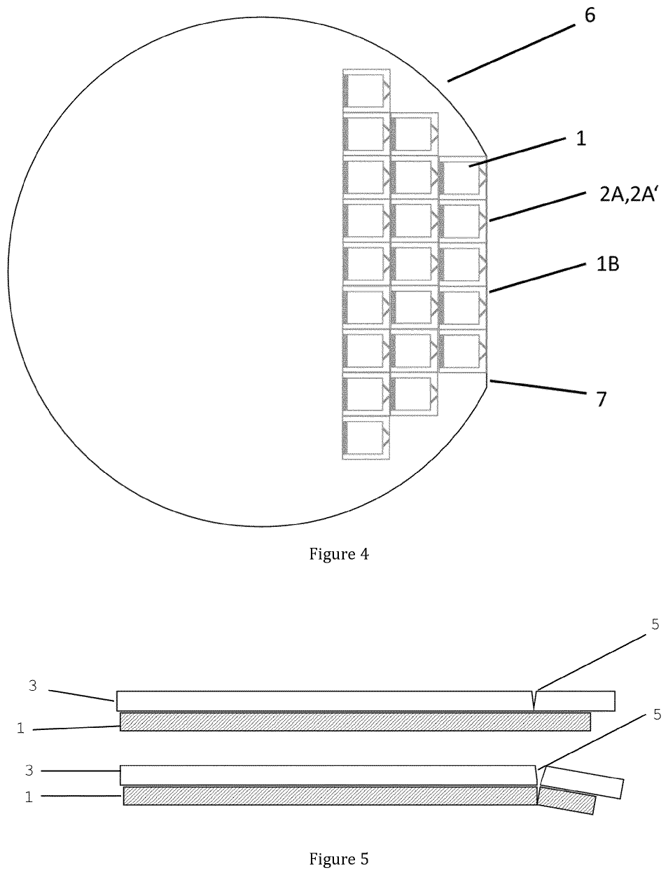

In preferred embodiments, the closing device covers the mono-crystalline wafer and comprises predetermined cleavage sites (5), which are aligned with the crystal orientation lines of the wafer. In this case, the pre-determined cleavage sites of the closing device may be suited to cleave the closing device and the microfluidic device of the wafer; see FIG. 5.

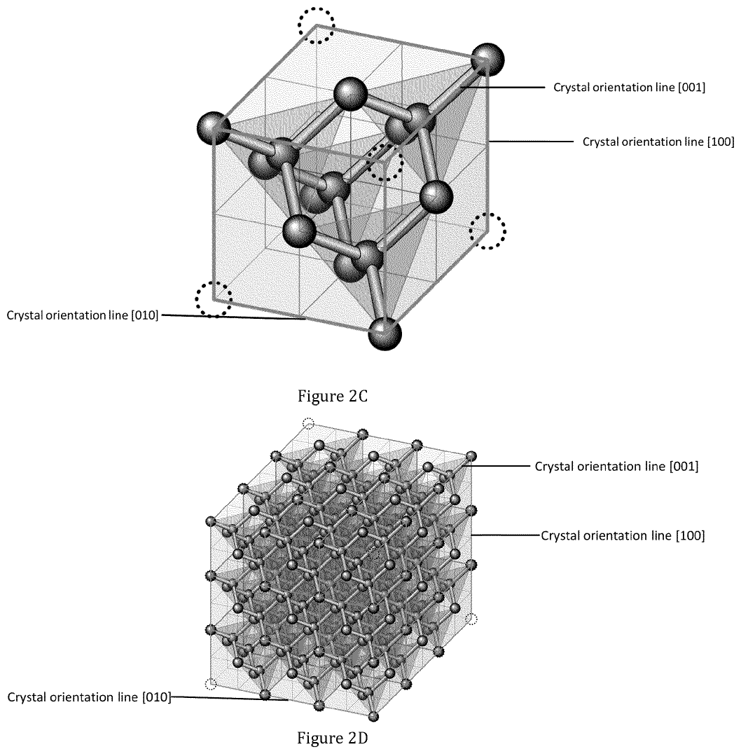

The method of the invention is particularly suitable for the production of microfluidic nozzles, preferably nozzles for use in an inhalation device, in particular impingement-type nozzles. An exemplified impingement-type nozzle is shown in FIG. 3.

In impingement-type nozzles, a fluid is ejected through two ejection channels (2, 2′) which each have a channel exit (2A,2A′) at a front end of the nozzle. The channels and channel exits are arranged as to eject liquid along respective ejection trajectories which intersect with one another at a collision point. The collision of the two fluid streams at the collision point generates a respirable aerosol.

To provide an ideal respirable aerosol, the ejection trajectories of the exit channels and the distance must be precisely defined. If the nozzle is produced according to the method of the invention, and the ejection channels are aligned with the crystal orientation line, the amount of reject nozzles can be reduced, as the number of nozzles unusable due to incorrect cutting or sawing is reduced.

Accordingly, in a particular embodiment, the invention relates to a method for producing a microfluidic device as defined above, wherein the microfluidic device is a nozzle for an inhalation device for nebulizing a liquid into a respirable aerosol, with a nozzle body (1) which has a front end (1B) and wherein the at least two microfluidic structures are ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein
the nozzle body (1) has a flat side (1A), with the at least two liquid channels (2, 2′) being entrenched with a defined depth (D) on said flat side (1A), and which has a front end (4B) that is, in a view perpendicular to a longitudinal axis (X) of the nozzle body (1), congruent with the front end (1B) of the nozzle body (1),
wherein the nozzle body is made at least in part from a mono-crystalline material and that the channel exits of each ejection channel are positioned in line with the crystal orientation so that the collision point is on the longitudinal axis (X) of the nozzle body
the method comprising the steps of
   a) preparing a nozzle body (1) which has a front end (1B) and which comprises at least two ejection channels (2, 2′), each channel (2, 2′) having an channel exit (2A, 2A′), wherein the ejection channels (2, 2′) are arranged such as to eject liquid along respective ejection trajectories which intersect with one another at a collision point on the longitudinal axis (X), wherein the nozzle body (1) has a flat side (1A), with the at least two liquid channels (2, 2′) being entrenched with a defined depth (D) on said flat side (1A), comprising the following steps:
      (i) providing a wafer substrate of a mono-crystalline material;
      (ii) fabricating on one side (1A) of said substrate a nozzle body comprising at least two liquid channels (2, 2′), said channels (2, 2′) having a defined depth (D), wherein said liquid channels exits are aligned along the crystal orientation;
      (iii) separating said body (1) from the substrate by cleaving the wafer along the crystal orientation line;
   b) optionally covering said nozzle body (1) with a closure device (3). In some embodiments of the invention, the channel exits are fabricated on the crystal orientation line and such as to eject liquid along respective ejection trajectories which intersect with one another at a collision point on the longitudinal axis (X). Preferably, the channel exits are each equidistant to the longitudinal axis.

In particular the method is suitable for producing a plurality of microfluidic nozzles as defined above.

In preferred embodiments, the nozzle is covered by a closing device, preferably a lid. In some embodiments, the closing device is a cover. The closing device may be of the same or different material as the nozzle. In some embodiments, the closing device is of a different material. In particular embodiments, the closing device is made of glass.

In some embodiments, the monocrystalline wafer is covered with a lid wafer, and the wafer is cleaved together with the lid wafer. In preferred embodiments, the cleaved lid wafer is kept on the cleaved nozzles to obtain nozzles covered with a closure device. In some embodiments, the lid wafer comprises predetermined breaking points, aligned with the crystal orientation lines of the mono-crystalline wafer and the nozzles fabricated thereon.

Nozzles produced with the method of the invention show are higher degree of precision and reproducibility of the aerosol quality.

In general, microfluidic devices produced with the method according to the invention require less material overall, as extra space that would be required as a safety measure to accommodate for inaccuracies can be reduced. Generally, the number of rejects of microfluidic devices can be reduced with the method.

In addition to the method, in a particular second aspect, the invention relates to a mono-crystalline wafer comprising a plurality of microfluidic devices as defined above, wherein the microfluidic devices each comprise at least two microfluidic structures, each of said structures is located at a front end of a microfluidic device; and wherein said front end of the microfluidic device and the microfluidic structures are aligned with the crystal orientation line. An example of such a wafer is shown in FIG. 4.

The mono-crystalline wafer comprising a plurality of microfluidic devices is preferably a wafer substrate cut from a monocrystal in crystal orientation and said wafer substrate comprises a flat side corresponding to a crystal orientation line.

The mono-crystalline wafer comprising a plurality of microfluidic devices according to this aspect of the invention is preferably a monocrystalline wafer obtained by cutting a mono-crystal from the Czochralski process. In particular, the mono-crystalline wafer comprising a plurality of microfluidic devices is a mono-crystal obtained from a material that crystallizes in the diamond or perovskite crystal structure. Suitable materials are known to the skilled person and include but are not limited to mono-crystalline silicone, mono-crystalline germanium or mono-crystalline gallium arsenide. In preferred embodiments the mono-crystalline material is mono-crystalline silicon.

In a preferred embodiment, said plurality of microfluidic devices are arranged in parallel orientations. Said plurality of microfluidic devices may be fabricated on the wafer by any suitable means as noted above. Suitable means include but are not limited to, etching, laser etching laser ablation, sand blasting and micro-lithography.

In a particular embodiment, said microfluidic devices comprised on the mono-crystalline wafer comprise a front end, a back end, and two side ends, wherein the devices are aligned that the front ends of the devices are aligned a first crystal orientation line, the back ends are aligned with a second crystal orientation line, parallel to the first crystal orientation line, and the side ends are aligned with crystal orientation lines perpendicular to the first and second crystal orientation line in such a way that the microfluidic devices may be separated by cutting or breaking along the crystal orientation lines.

In a particular embodiment, the plurality of microfluidic devices comprised on the mono-crystalline wafer is arranged in a checkerboard pattern, wherein the back end of one device is in contact with the front end of another device and the side ends of one. As such the wafer allow to obtain a plurality of microfluidic devices by cleaving the devices off along the respective crystal orientation lines. In particular, the microfluidic devices are obtained by breaking off the devices at the respective crystal orientation lines.

Therefore, in a further aspect, the invention relates to microfluidic devices obtainable by the method as described above. In particular, the invention relates to microfluidic nozzles obtainable by the method as defined above.

Accordingly, in one aspect the invention relates to a microfluidic device, comprising at least two microfluidic structures, each of said structures is located at a front end of the microfluidic device; characterized in that the microfluidic device is made at least in part from a mono-crystalline material and wherein said front end of the microfluidic device and the microfluidic structures are aligned with a crystal orientation line of the mono-crystalline material. Preferably, the front end of the device is on a crystal orientation line.

In particular, said front end of the microfluidic device follows or corresponds to a crystal orientation line of the mono-crystalline material. Most preferably, the front end of the microfluidic device is obtained by cleaving said device from a mono-crystalline wafer at a crystal orientation line.

The mono-crystalline material of the microfluidic device preferably corresponds to a mono-crystalline material obtained from a mono-crystal sliced in one crystal plane. More preferably, the mono-crystalline material is a mono-crystalline material obtained from a mono-crystal sliced in [100] crystal direction and the crystal orientation line to which the front end of the microfluidic device is aligned corresponds to a [010] or [001] crystal orientation line.

Aligning the microfluidic structures with the crystal orientation line allows for an easy and simple way to obtain reproducible microfluidic devices. Any type of microfluidic structure is suitable. Preferably, said microfluidic structures are fixed on the chip. Preferred microfluidic structures include but are not limited to microfluidic chambers or reservoirs, and microfluidic channels. Microfluidic structures also include specific structures, such as microfluidic filter structures. Microfluidic structures also include microfluidic structures which may comprise additional components, which are not part of the mono-crystalline wafer substrate, such as microfluidic valves or mixing chambers.

In particular, microfluidic structures comprising microfluidic channels with microfluidic channel exits are preferred. An alignment along the crystal orientation line allows for precise positioning and when the device is cleaved along said crystal orientation line, said precise positioning is not affected by e.g., imprecise cutting or sawing. Preferably, said microfluidic structures are present, e.g., etched into, the monocrystalline material.

The skilled person is aware of suitable mono-crystalline materials. Preferably, the mono-crystalline material is a material that crystallizes in the diamond crystal structure or a perovskite crystal structure. Preferred materials for the microfluidic device include mono-crystalline silicone, mono-crystalline germanium or mono-crystalline gallium arsenide. In preferred embodiments the mono-crystalline material is mono-crystalline silicon.

The microfluidic device according to the present invention preferably comprises a front end and a back end. The microfluidic device further comprises two or more side ends, however it is preferred that the device comprises two side ends and is in general of rectangular shape. However, the microfluidic device is not limited to a rectangular shape.

Mono-crystalline materials allow for small and compact microfluidic devices with a reduced thickness. In some embodiments, the microfluidic device according to the invention, has a thickness of about 1.5 mm or less. In preferred embodiments, the microfluidic device is less than 1.5 mm thick, for example it has a thickness of about 250 μm to about 1.5 mm. In some embodiments the thickness is about 1 mm or less. In preferred embodiments, the thickness is about 750 μm or less, e.g., between about 300 μm and about 750 μm.

The at least two microfluidic structures preferably comprise microfluidic channels which comprise microfluidic channel exits. Said channel exits can be positioned with high precision, which ensures compatibility with other devices, e.g., for transferring fluids from one device to another. A microfluidic device according to the invention shows a high precision compared to a microfluidic device obtained by cutting or sawing, and thus the number of devices which are incompatible due to misaligned cutting or sawing is reduced.

In preferred embodiments, the microfluidic device comprises microfluidic channels and microfluidic channel exits and said microfluidic channel exits are located at the front end of the microfluidic device and aligned with the crystal orientation line.

The microfluidic device may be any type of microfluidic device. In some embodiments, the microfluidic device is an open microfluidic device. In some embodiments the microfluidic device is at least partly closed. In some embodiments, the microfluidic device comprises a closing device, covering at least part of the device. In some embodiments, the device comprises a closing device covering the microfluidic structures. In some embodiments the microfluidic device comprises a closing device covering parts or the whole device. Said closing device may be a lid. In some embodiments said closing device is a cover.

If the device comprises a closing device, the closing device may be made from any material. In some embodiments the closing device is made from the same mono-crystalline material. In some embodiments the closing device is made from a different material. Any material suitable to be used in microfluidic applications may be used. Suitable materials are known to the skilled person. In some embodiments, wherein the microfluidic device comprises a closing device, the closing is made from glass.

The microfluidic device according to the present invention may be any microfluidic device which requires a high degree of precision. In preferred embodiments of the invention, the microfluidic device is for an inhalation device. In some embodiments, the microfluidic device is or comprises a valve or is part of a valve. In other embodiments, the microfluidic device is or comprises a filter unit. In some embodiments, the microfluidic device according to the invention is a nozzle for an inhalation device. In particular embodiments, the microfluidic device according to the invention is an impingement type nozzle.

Accordingly, in a particular embodiment, the invention relates to a microfluidic nozzle, wherein the nozzle is a nozzle for an inhalation device for nebulizing a liquid into a respirable aerosol, with a nozzle body (1) which has a front end (1B) and wherein the at least two microfluidic structures are ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein
the nozzle body (1) has a flat side (1A), with the at least two liquid channels (2, 2′) being entrenched with a defined depth (D) on said flat side (1A), and which has a front end (4B) that is, in a view perpendicular to a longitudinal axis (X) of the nozzle body (1), congruent with the front end (1B) of the nozzle body (1),
characterized in that the nozzle body is made from a mono-crystalline material and that the channel exits of each ejection channel are positioned in line with the crystal orientation so that the collision point is on the longitudinal axis of the nozzle body.

In preferred embodiments of the invention, the microfluidic channel exits of the ejection channels are on a crystal orientation line of the mono-crystalline material.

The mono-crystalline material of the nozzle preferably corresponds to a mono-crystalline material obtained from a mono-crystal sliced in one crystal plane. More preferably, the mono-crystalline material is a mono-crystalline material obtained from a mono-crystal sliced in [100] crystal direction and the crystal orientation line to which the front end of the microfluidic device is aligned corresponds to a [010] or [001] crystal orientation line.

In some embodiments, the nozzle comprises a closing device, preferably a lid. In some embodiments, said closing device is a cover. Said closing device may be made from the same material as the nozzle or of a different material. In some embodiments, the closing device is made from glass.

The invention further relates to the use of a microfluidic device obtained by a method as defined above as a nozzle in an inhalation device for nebulizing a liquid into a respirable aerosol.

In a particular aspect, the invention further relates to an Inhalation device for nebulizing a liquid into a respirable aerosol comprising at least one microfluidic device as defined above, in particular a nozzle as defined above.

All embodiments as described above in connection with the method of the present invention apply to all aspects of the invention, including the microfluidic device according to the invention as well as the mono-crystalline wafer according to the invention.

The invention further relates to the following numbered items:

1. Microfluidic device, comprising at least two microfluidic structures, each of said structures is located at a front end of the microfluidic device; characterized in that the microfluidic device is made at least in part from a mono-crystalline material and wherein said front end of the microfluidic device and the microfluidic structures are aligned with a crystal orientation line of the monocrystalline material.

2. Microfluidic device according to item 1, wherein said microfluidic structures are microfluidic channels comprising microfluidic channel exits.

3. Microfluidic device according to item 2, wherein said microfluidic channel exits are located at the front end of the microfluidic device and aligned with the crystal orientation line.

4. Microfluidic device according to any one of items 1 to 3, wherein the microfluidic device is made from a mono-crystalline material, wherein the material crystallizes in diamond cubic crystal structure or a perovskite crystal structure.

5. Microfluidic device according to any one of items 1 to 4, wherein the mono-crystalline material is selected from silicone, germanium and gallium arsenide.

6. Microfluidic device according to any one of items 1 to 5, wherein the microfluidic structures are present in the mono-crystalline material.

7.-Microfluidic device according to any one of items 1 to 6 wherein the microfluidic device comprises a closing device.

8. Microfluidic device according to item 7, wherein the closing device is made from the same mono-crystalline material.

9. Microfluidic device according to item 7, wherein the closing device is made from a different material.

10. Microfluidic device according to item 9, wherein the closing device is made from glass.

11. Microfluidic device according to any one of items 1 to 10, wherein the microfluidic device is a nozzle.

12. Microfluidic device according to item 11, wherein said nozzle is an impingement-type nozzle.

13. Microfluidic device according to item 12, wherein the nozzle is a nozzle for an inhalation device for nebulizing a liquid into a respirable aerosol, with a nozzle body (1) which has a front end (1B) and wherein the at least two microfluidic structures are ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein
the nozzle body (1) has a flat side (1A), with the at least two liquid channels (2, 2′) on said flat side (1A), and which has a front end (3B) that is, in a view perpendicular to a longitudinal axis (X) of the nozzle body (1), congruent with the front end (1B) of the nozzle body (1),
characterized in that the nozzle body is made from a mono-crystalline material and that the channel exits of each ejection channel are positioned in line with the crystal orientation so that the collision point is on the longitudinal axis of the nozzle body.

14. Microfluidic device according to item 13, wherein the nozzle comprises a closing device.

15. Mono-crystalline wafer comprising a plurality of microfluidic devices according to any one of items 1 to 14.

16. Mono-crystalline wafer according to item 15, wherein the plurality of microfluidic devices are arranged in parallel orientations.

17. Mono-crystalline wafer according to item 16, wherein the microfluidic devices comprise a front end, a back end, and two side ends, wherein the devices are aligned that the front ends of the devices are aligned a first crystal orientation line, the back ends are aligned with a second crystal orientation line, parallel to the first crystal orientation line, and the side ends are aligned with crystal orientation lines perpendicular to the first and second crystal orientation line in such a way that the microfluidic devices may be separated by cutting or breaking along the crystal orientation lines.

18. Monocrystalline wafer according to item 17, wherein the plurality of microfluidic devices is arranged in a checkerboard pattern, wherein the back end of one device is in contact with the front end of another device and the side ends of one device are in contact with side ends of other devices.

19. Method for producing a microfluidic device according to any one of items 1 to 14, comprising the steps of
a) providing a wafer substrate of a mono-crystalline material;
b) fabricating on said wafer substrate at least one microfluidic device comprising at least two microfluidic structures, wherein the microfluidic structures are aligned along the crystal orientation;
c) separating said microfluidic device (1) from the substrate by cleaving the wafer along the crystal orientation line.

20. Method for producing a microfluidic device according to item 19, wherein the method comprises etching the microfluidic structures of said device into the wafer.

21. Method for producing a microfluidic device, wherein the microfluidic device is a nozzle for an inhalation device according to any one of items 12 to 14, the method comprising the steps of
a) preparing a nozzle body (1) which has a front end (1B) and which comprises at least two ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein at least one recess (3) is provided at the front end (1B) in which at least two of the channel exits (2A, 2A′) are positioned, wherein the nozzle body (1) has a flat side (1A), with the at least two liquid channels (2, 2′) being entrenched with a defined depth (D) on said flat side (1A), comprising the following steps:
(i) providing a wafer substrate of a mono-crystalline material;
(ii) fabricating on one side (1A) of said substrate at least two liquid channels (2, 2′), said channels (2, 2′) having a defined depth (D), wherein said liquid channels exits are aligned along the crystal orientation;
(iii) separating said body (1) from the substrate by cleaving the wafer along the crystal orientation line;
b) optionally covering said nozzle body (1) with a closing device.

22. Nozzle for an inhalation device obtainable by a method according to item 21.

23. Use of a microfluidic device according to any one of items 12 to 14 as a nozzle in an inhalation device for nebulizing a liquid into a respirable aerosol.

24. Inhalation device for nebulizing a liquid into a respirable aerosol comprising at least one microfluidic device according to any one of items 1 to 14.

### LIST OF REFERENCES

1 nozzle body
1A flat side
1B front end
2, 2′ ejection channel, liquid channel, channel
2A, 2A′ channel exit
2B, 2B′ front end channel exit
3 closing device
4 separation line
4′ optimal separation line
5 pre-determined cleavage site
6. Mono-crystalline wafer
7. flat side of the wafer
A, A′ jet axis
X longitudinal axis
Y, Y′ lateral distance
D depth

1. Microfluidic device, comprising at least two microfluidic structures, each of said structures is located at a front end of the microfluidic device; characterized in that the microfluidic device is made at least in part from a mono-crystalline material and wherein said front end of the microfluidic device and the microfluidic structures are aligned with a crystal orientation line of the monocrystalline material; wherein the microfluidic device is a nozzle for an inhalation device.

2. Microfluidic device according to claim 1, wherein said microfluidic structures are microfluidic channels comprising microfluidic channel exits, wherein said microfluidic channel exits are located at the front end of the microfluidic device and aligned with the crystal orientation line.

3. Microfluidic device according to any one of claim 1 or 2, wherein the microfluidic device is made from a mono-crystalline material, wherein the material crystallizes in diamond cubic crystal structure or a perovskite crystal structure.

4. Microfluidic device according to any one of claims 1 to 3, wherein the microfluidic device is made from a mono-crystalline material, wherein the mono-crystalline material is selected from silicone, germanium and gallium arsenide.

5. Microfluidic device according to any one of claims 1 to 4, wherein the microfluidic device comprises a closing device.

6. Microfluidic device according to claim 5, wherein the closing device is made from glass.

7. Microfluidic device according to any one of claims 1 to 6, wherein the nozzle is an impingement-type nozzle.

8. Microfluidic device according to claim 7, wherein the nozzle is a nozzle for an inhalation device for nebulizing a liquid into a respirable aerosol, with a nozzle body (1) which has a front end (1B) and wherein the at least two microfluidic structures are ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein
the nozzle body (1) has a flat side (1A), with the at least two liquid channels (2, 2′) on said flat side (1A), and which has a front end (4B) that is, in a view perpendicular to a longitudinal axis (X) of the nozzle body (1), congruent with the front end (1B) of the nozzle body (1),
and wherein the nozzle body is made from a mono-crystalline material having a crystal orientation line and wherein the channel exits of each ejection channel are positioned in line with the crystal orientation so that the collision point is on the longitudinal axis of the nozzle body.

9. Mono-crystalline wafer comprising a plurality of microfluidic devices according to any one of claims 1 to 8, wherein the microfluidic devices are nozzles for an inhalation device.

10. Mono-crystalline wafer according to claim 9, wherein the wafer comprises
a first crystal orientation line,
a second crystal orientation line, parallel to the first crystal orientation line, and
a plurality of crystal orientation lines perpendicular to the first and second crystal orientation line, and
wherein the microfluidic devices comprise
a front end,
a back end,
two side ends,
wherein the devices are aligned so that the front ends of the devices are aligned to the first crystal orientation line of the wafer, the back ends are aligned with the second crystal orientation line of the wafer, and the side ends are aligned with the crystal orientation lines of the wafer perpendicular to the first and second crystal orientation.

11. Mono-crystalline wafer according to claim 9 or 10, wherein the plurality of microfluidic devices is arranged in a checkerboard pattern, wherein the back end of one device is in contact with the front end of another device and the side ends of one device are in contact with side ends of other devices.

12. Method for producing a microfluidic device, wherein the microfluidic device is a nozzle for an inhalation device according to any one of claims 6 to 8, the method comprising the steps of
a) preparing a nozzle body (1) which has a front end (1B) and which comprises at least two ejection channels (2, 2′), each channel (2, 2′) having an channel exit (2A, 2A′), wherein the ejection channels (2, 2′) are arranged such as to eject liquid along respective ejection trajectories which intersect with one another at a collision point, wherein at least one recess (3) is provided at the front end (1B) in which at least two of the channel exits (2A, 2A′) are positioned, wherein the nozzle body (1) has a flat side (1A), with the at least two liquid channels (2, 2′) being entrenched with a defined depth (D) on said flat side (1A), comprising the following steps:
(i) providing a wafer substrate of a mono-crystalline material having a crystal orientation line;
(ii) fabricating on one side (1A) of said substrate at least two liquid channels (2, 2′), said channels (2, 2′) having a defined depth (D), wherein said liquid channels exits are aligned along the crystal orientation line;
(iii) separating said body (1) from the wafer substrate by cleaving the wafer along the crystal orientation line;
b) optionally covering said nozzle body (1) with a closing device.

13. Method according to claim 11 or 12, wherein the step of separating the microfluidic device from the wafer substrate is performed by breaking off the microfluidic device from the wafer substrate.

14. Use of a microfluidic device according to any one of claims 6 to 8 as a nozzle in an inhalation device for nebulizing a liquid, preferably a medically active liquid, into a respirable aerosol.

15. Inhalation device for nebulizing a liquid into a respirable aerosol comprising at least one microfluidic device according to any one of claims 1 to 8.
